(19) United States
(12) Patent Application Publication
(10) Pub. No.: US 2020/0323426 A1
(43) Pub. Date: Oct. 15, 2020

(54) IN-VIVO INSERTABLE DEVICES HAVING FAIL-SAFE EVACUATION

(71) Applicant: MI FITPILL MEDICAL LTD., Haifa (IL)

(72) Inventors: Menachem Peter WEISS, Haifa (IL); Yaakov GREENBERG, Even Yehuda (IL); Gabriel SINBAR, Timrat (IL); Abraham LEVANON, Haifa (IL)

(73) Assignee: MI FITPILL MEDICAL LTD., Haifa (IL)

(21) Appl. No.: 16/955,237

(22) PCT Filed: Dec. 25, 2018
(86) PCT No.: PCT/IL18/51387

§ 371 Date: Jun. 18, 2020

Related U.S. Application Data
(60) Provisional application No. 62/709,015, filed on Jan. 3, 2018.

(51) Int. Cl. A61B 1/24; A61B 1/005; A61B 1/04; A61B 1/00

(52) U.S. Cl. CPC A61B 1/24; A61B 1/0058; A61B 1/00133; A61B 1/00016; A61B 1/00006; A61B 1/041

(57) ABSTRACT

An in-vivo insertable device, which is insertable into an at least partially hollow organ of a human or animal, including a housing formed of a material and being of a contour which enable it to be inserted into an at least partially hollow organ of a human or animal, an operative portion disposed within the housing, at least one device-retaining element having three operational states: a first retracted state prior to and during insertion of the device, a second expanded state operative to retain the operative portion within the at least partially hollow organ and a third disassembled state that enables the device to be naturally evacuated from the at least partially hollow organ, the in-vivo insertable device including at least two mutually redundant, mutually diverse and mutually independently operative disassembly mechanisms which cause the at least one device-retaining element to shift to the third disassembled state.

### REFERENCE TO RELATED APPLICATIONS

Reference is hereby made to U.S. Provisional Patent Application Ser. No. 62/709,015, filed Jan. 3, 2018 and entitled FAIL SAFE EVACUATION OF IN-VIVO INSERTED DEVICES, the disclosure of which is hereby incorporated by reference and priority of which is hereby claimed pursuant to 37 CFR 1.78(a) (1). Reference is also made to applicant's U.S. Pat. Nos. 8,021,384 and 9,780,622, the disclosures of which are hereby incorporated by reference.

### FIELD OF THE INVENTION

The present invention relates to in-vivo insertable devices generally and more particularly to in-vivo insertable devices which include disassembly mechanisms.

### BACKGROUND OF THE INVENTION

Various types of in-vivo devices and their disassembly mechanisms are known in the literature.

### SUMMARY OF THE INVENTION

The present invention seeks to provide improved in-vivo insertable devices which include disassembly mechanisms.

There is thus provided in accordance with a preferred embodiment of the present invention, an in-vivo insertable device, which is insertable into an at least partially hollow organ of a human or animal, including a housing formed of a material and being of a contour which enable it to be inserted into an at least partially hollow organ of a human or animal, an operative portion disposed within the housing, at least one device-retaining element having three operational states: a first retracted state prior to and during insertion of the device, a second expanded state operative to retain the operative portion within the at least partially hollow organ and a third disassembled state that enables the device to be naturally evacuated from the at least partially hollow organ, the in-vivo insertable device being characterized in that it includes at least two mutually redundant, mutually diverse and mutually independently operative disassembly mechanisms which cause the at least one device-retaining element to shift to the third disassembled state.

Preferably, the at least one device-retaining element includes at least two device-retaining elements and the at least two mutually redundant, mutually diverse and mutually independently operative disassembly mechanisms cause the at least two device-retaining elements to shift from the second expanded state to the third disassembled state simultaneously.

In accordance with a preferred embodiment of the present invention the at least one device-retaining element includes a first remotely actuable mechanism and a second automatically operable mechanism. Additionally, the second automatically operable mechanism is one of a biodegradable, bioabsorbable and bioresorbable element which biodegrades within the organ and thereby causes the shift to the third disassembled state.

In accordance with a preferred embodiment of the present invention the first remotely actuable mechanism includes an electromagnetic actuator. Alternatively, the first remotely actuable device includes a heat responsive shape changing element.

Preferably, the first remotely operable mechanism is actuatable by a remote controller outside of the human or animal.

In accordance with a preferred embodiment of the present invention the second automatically operable mechanism is actuable by a controller included within the operative portion in accordance with a preplanned program.

In accordance with a preferred embodiment of the present invention the first remotely actuable device includes a heat responsive displaceable element having a shape memory, which undergoes heating in response to remote actuation. Additionally, in the first remotely actuable mechanism the heating is achieved by connecting electric contacts directly to the heat responsive displaceable element having a shape memory, which serves also as a heating element.

In accordance with a preferred embodiment of the present invention at least one of the at least two mutually redundant, mutually diverse and mutually independently operative disassembly mechanisms employ at least one of magnetic forces, electric current, electrostatic forces, external pressure and a heating element.

Preferably, the heating is triggered by a controller and achieved by connecting batteries, located in the payload, to the heat responsive displaceable element.

In accordance with a preferred embodiment of the present invention all dissembled elements of the device are formed with rounded and smooth edges so that they will not harm any parts of a hollow organ during evacuation.

In accordance with a preferred embodiment of the present invention at least one of the at least two mutually redundant, mutually diverse and mutually independently operative disassembly mechanisms can be actuated via endoscopy.

In accordance with a preferred embodiment of the present invention at least one of the at least two mutually redundant, mutually diverse and mutually independently operative disassembly mechanisms includes at least one of a split band and a connection part which holds the device retaining elements onto the main element, and which is operative when the at least one mechanism is actuated to release the device retaining elements, so that the device and all its parts are free to evacuate the hollow organ.

In accordance with a preferred embodiment of the present invention the heat responsive displaceable element is heated by at least one of a heating micro-wire and a heating sheet. Additionally, the at least one of a heating micro-wire and a heating sheet is covered by flexible insulation cover made of one or more of plastic, rubber, shrink tubing and vacuum deposited polymer coating.

There is also provided in accordance with another preferred embodiment of the present invention an in-vivo insertable device, which is insertable into an at least partially hollow organ of a human or animal, including a housing formed of a material and being of a contour which enable it to be inserted into an at least partially hollow organ of a human or animal, an operative portion disposed within the housing, at least one device-retaining element having three operational states: a first retracted state prior to and during insertion of the device, a second expanded state operative to retain the operative portion within the at least partially hollow organ and a third disassembled state that enables the device to be naturally evacuated from the at least partially hollow organ, the in-vivo insertable device being characterized in that it includes at least two mutually redundant and mutually independently operative disassembly mechanisms which cause the at least one device-retaining element to shift to the third disassembled state.

### BRIEF DESCRIPTION OF THE DRAWINGS

The present invention will be understood and appreciated more fully from the description which follows with reference to the drawings in which:

FIGS. 1A, 1B and 1C are simplified respective assembled, sectional and exploded view illustrations of an embodiment of an in-vivo insertable device constructed and operative in accordance with a preferred embodiment of the present invention, FIG. 1B being taken along lines 1B-1B in FIG. 1A;

FIGS. 2A, 2B, 2C and 2D are simplified illustrations of the device of FIGS. 1A-1C in the following operative orientations: immediately post insertion within an organ, such as the stomach; post insertion and fully deployed within an organ; partially disassembled using a first disassembly mechanism and partially disassembled using a second disassembly mechanism;

FIGS. 3A, 3B and 3C are simplified respective assembled, sectional and exploded view illustrations of another embodiment of an in-vivo insertable device constructed and operative in accordance with a preferred embodiment of the present invention, FIG. 3B being taken along lines 3B-3B in FIG. 3A;

FIGS. 4A, 4B, 4C and 4D are simplified illustrations of the device of FIGS. 3A-3C in the following operative orientations: immediately post insertion within an organ, such as the stomach; post insertion and fully deployed within an organ; partially disassembled using a first disassembly mechanism and partially disassembled using a second disassembly mechanism;

FIGS. 5A, 5B and 5C are simplified respective assembled, sectional and exploded view illustrations of yet another embodiment of an in-vivo insertable device constructed and operative in accordance with a preferred embodiment of the present invention, FIG. 5B being taken along lines 5B-5B in FIG. 5A; and

FIGS. 6A, 6B, 6C and 6D are simplified illustrations of the device of FIGS. 5A-5C in the following operative orientations: immediately post insertion within an organ, such as the stomach; post insertion and fully deployed within an organ; partially disassembled using a first disassembly mechanism and partially disassembled using a second disassembly mechanism.

### DETAILED DESCRIPTION OF PREFERRED EMBODIMENTS

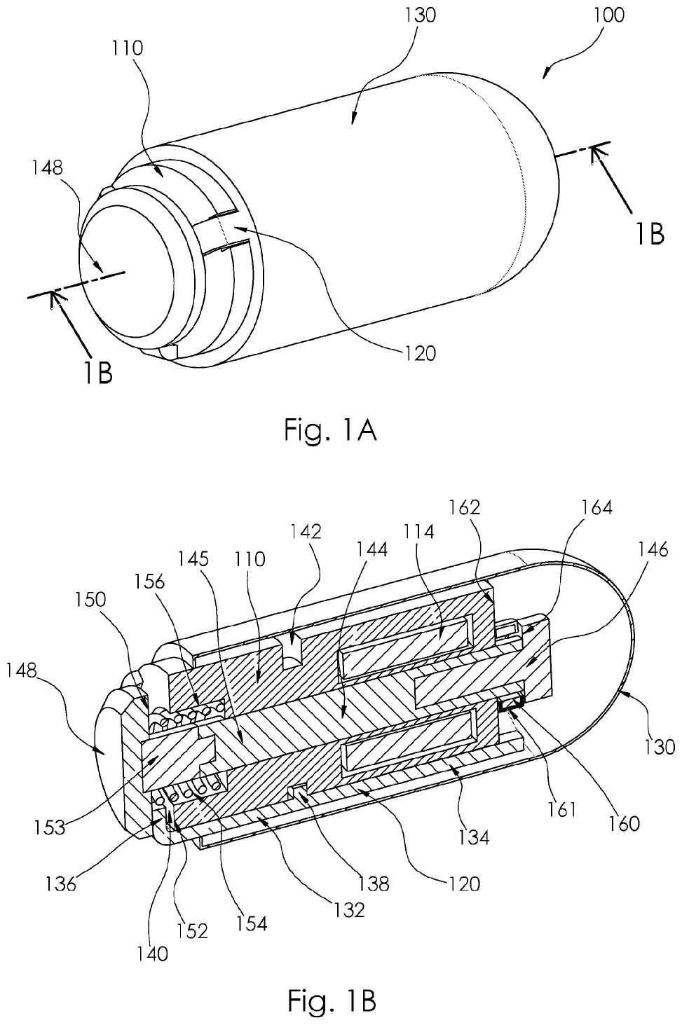

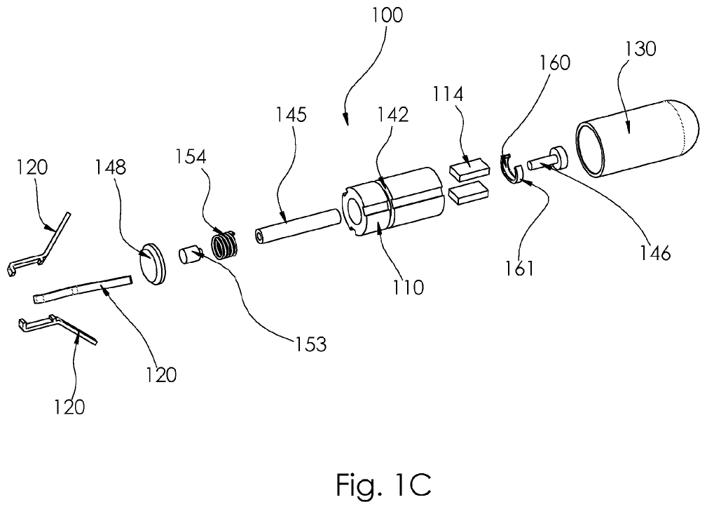

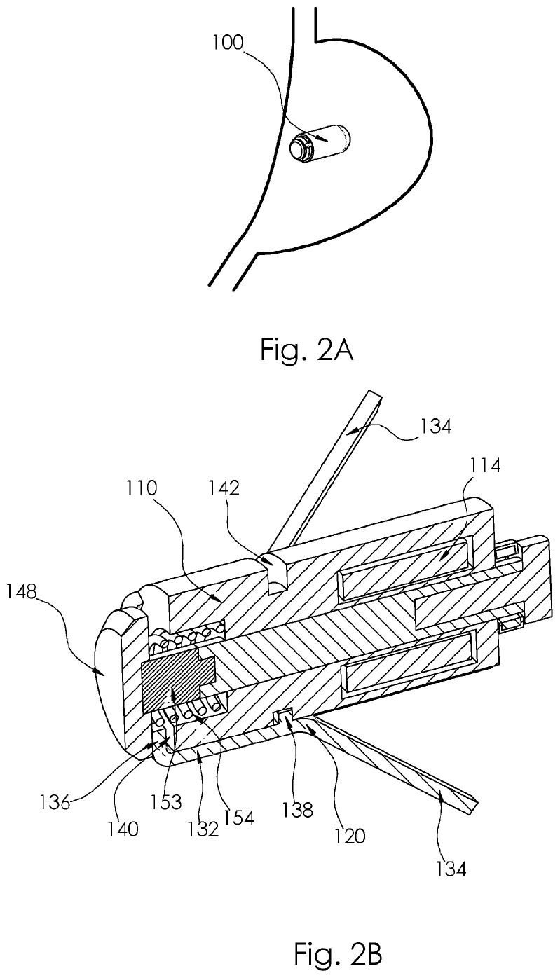

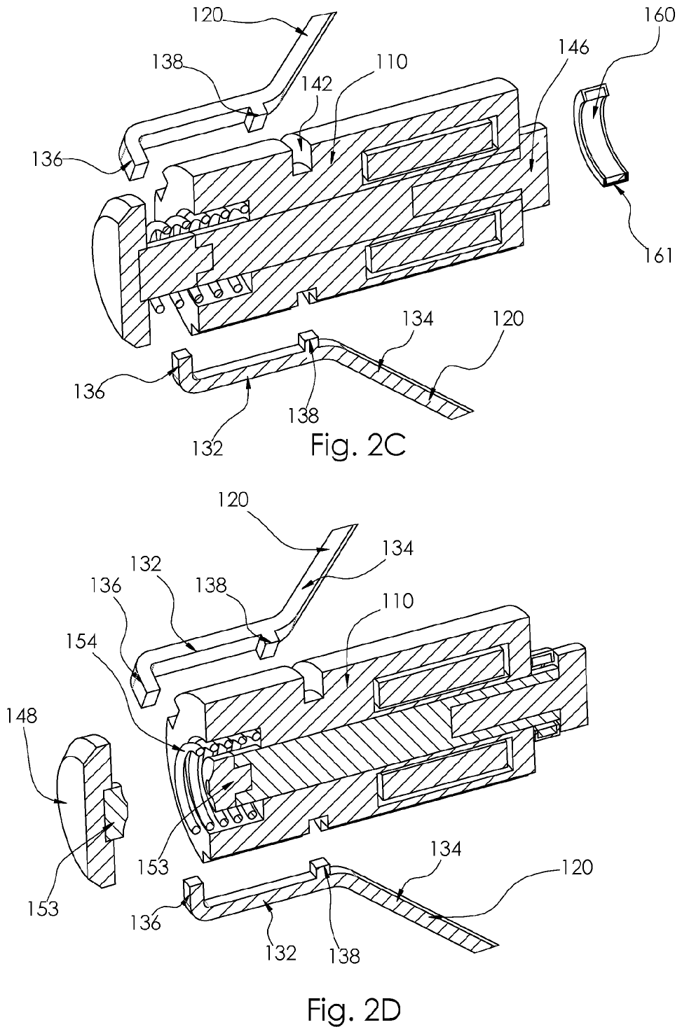

Reference is now made to FIGS. 1A, 1B and 1C, which are simplified respective assembled, sectional and exploded view illustrations of an embodiment of an in-vivo insertable device 100 constructed and operative in accordance with a preferred embodiment of the present invention. For clarity, reference is also made, in the course of the description which follows, to FIGS. 2A-2D.

It is to be appreciated that the in-vivo insertable device 100 may be any suitable in-vivo insertable device 100, preferably in the form of a capsule, which is suitable for insertion into an at least partially hollow organ of a human or animal and is intended for subsequent evacuation therefrom. Examples of suitable in-vivo insertable devices 100 include devices for drug perfusion, devices for in-vivo chemical analysis and devices for gastrointestinal tonometry. The description which follows refers generally to an in-vivo insertable device 100 which is particularly suitable for insertion into a human stomach, it being understood that the present invention is not limited to this example. Reference is also made in this connection to applicant's U.S. Pat. Nos. 8,021,384 and 9,780,622, the disclosures of which are hereby incorporated by reference.

Turning now to FIGS. 1A-1C, it is seen that in-vivo insertable device 100 preferably includes a main portion 110, which contains a payload 114, preferably comprising operative elements of the device 100. Examples of such operative elements include: electronics, batteries, motors, piezoelectric elements, controllers and wireless communication assemblies.

A plurality of device retaining elements 120 are preferably removably mounted onto main portion 110 and are retained in a retracted operative orientation by a capsule cover element 130, typically a gelatin capsule cover element which biodegrades upon being located within an organ, such as a stomach. FIG. 2A shows the device 100 just following location thereof within the stomach and prior to biodegrading of capsule cover element 130.

Device retaining elements 120 preferably each include a main portion 132 and a wing portion 134 which is pre-stressed relative to the main portion 132, so as to assume the bent orientation shown in FIG. 2B, but is constrained by the capsule cover element 130, when intact, to assume the straight orientation shown in FIG. 1B.

Device retaining elements 120 also preferably include a forward inwardly directed retaining tab 136 and a rearward inwardly directed retaining tab 138, which are seated respectively at a location 140 forwardly of main portion 110 and in a circumferential slot 142 formed in main portion 110, in the operative orientations shown in FIGS. 2A and 2B. Reference to “forward” or “forwardly” in this description refers, in FIGS. 1A-2D, to the left in these drawings.

Upon degradation of the capsule cover element 130, the wing portions 134 of device retaining elements 120 automatically pivot outwardly from the main portion 110. The automatic pivoting preferably occurs due to pre-stressing of the device retaining elements 120, which are held in their retracted operative orientation by capsule cover element 130. FIG. 2B shows the device in an operative orientation wherein the device 100 is retained within the organ by the spreading out of the device retaining elements 120, resulting in an increased size of the device.

It is a particular feature of a preferred embodiment of the present invention that the device 100 is provided with a fail-safe evacuation assembly 144, including mutually redundant, mutually diverse and mutually independently operative mechanisms which enable evacuation of the device 100 from the organ by disassembly of the device retaining elements 120 from the main portion 110.

In accordance with a preferred embodiment of the present invention shown in FIGS. 1A-2D, the fail-safe evacuation assembly 144 comprises a retaining rod sub-assembly, preferably including a forward rod portion 145 and a rearward rod portion 146, which partially extends through forward rod portion 145 from the rear thereof.

Forward of forward rod portion 145 there is preferably provided a retaining head portion 148, defining a rearward-facing surface 150, which engages forward tabs 136 of device retaining elements 120 and retains them in tight engagement facing a corresponding forward-facing surface 152 of main portion 110, thus retaining them against disengagement from main portion 110 in the operative orientations shown in FIGS. 2A and 2B.

Preferably, retaining head portion 148 is connected to forward rod portion 145 by a biodegradable connection portion 153, preferably made of a biodegradable material such as PLGA, PLA, PGA and others known to the art. This biodegradable connection portion provides one element of the fail-safe evacuation assembly 144.

A compression spring 154 is preferably seated in a circumferential recess 156 formed in a forward end of main portion 110 and engages rearward-facing surface 150 of retaining head portion 148. Compression spring 154 urges retaining rod sub-assembly, including forward rod portion 145, rearward rod portion 146, retaining head portion 148 and biodegradable connection portion 153, forwardly, i.e. to the left in FIGS. 1A-2D.

Forward displacement of the retaining rod sub-assembly relative to the main portion 110 is prevented, however, in the operative orientations of FIGS. 2A and 2B, by the provision of a remotely removable retaining element, such as a split nitinol ring 160, at least partially covered by a resistance heating wire 161, both of which are seated between a rearward-facing surface 162 of main portion 110 and a forward-facing surface 164 of rearward rod portion 146.

It is appreciated that the remotely removable retaining element provides another element of the fail-safe evacuation assembly 144, inasmuch as it can be disengaged from its location by heating of resistance heating wire 161 which may be actuated by a remote control and powered via the payload 114.

Upon disengagement of the remotely removable retaining element, spring 154 shifts the retaining rod sub-assembly forwardly relative to main portion 110 and thus releases retaining tabs 136 and 138 of device retaining element 120 from engagement with main portion 110 as seen in FIG. 2C. The same result is achieved in a redundant but different manner by degradation of biodegradable connection 153, which decouples retaining head portion 148 from the remainder of the retaining rod assembly and thus releases retaining tabs 136 and 138 of device retaining elements 120 from engagement with main portion 110, as seen in FIG. 2D.

As seen clearly in both FIGS. 2C and 2D, this disengagement enables total disengagement of device retaining elements 120 from main portion 110 and enables the device retaining elements 120 and the main portion 110 as well as the various elements of the fail-safe evacuation assembly 144 to be readily evacuated from the organ, such as the stomach, by natural, biological evacuation processes, inasmuch as the size of each of the various elements is substantially smaller than that of the device in its operative orientation shown in FIG. 2B. It is a particular feature of embodiments of the present invention that disengagement of the retaining head portion 148, by either of the two fail safe mechanisms, releases all of the retaining tabs 136 and 138 simultaneously, so all device retaining elements 120 are also released simultaneously. This is important to prevent possible piercing of an organ wall by a device retaining element 120 which remains attached to the main portion 110 during evacuation of the device from the organ.

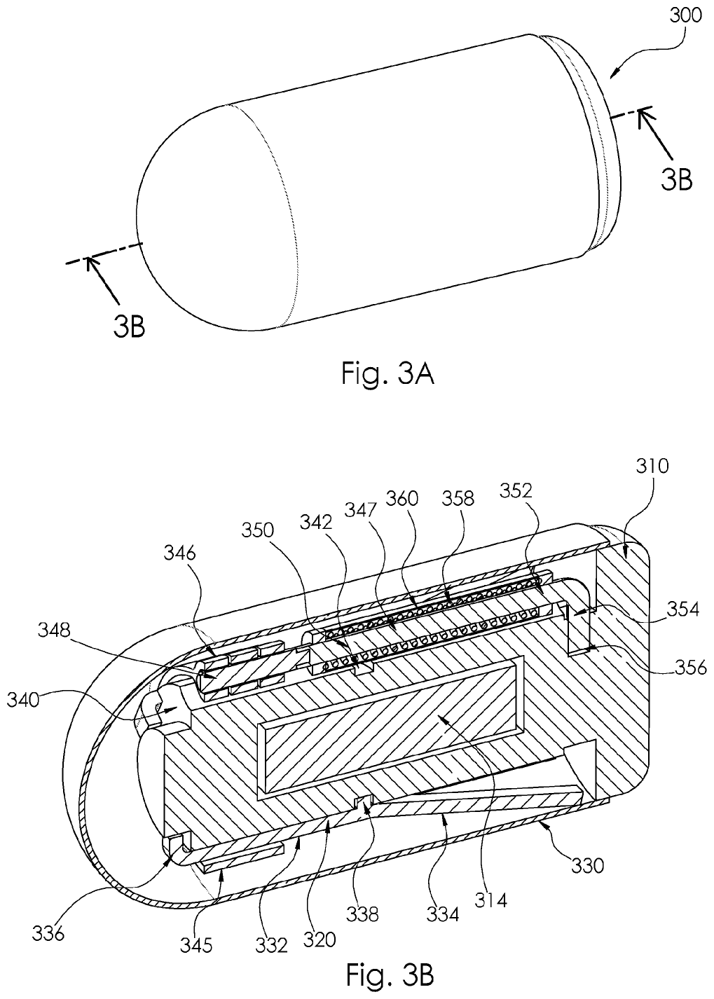

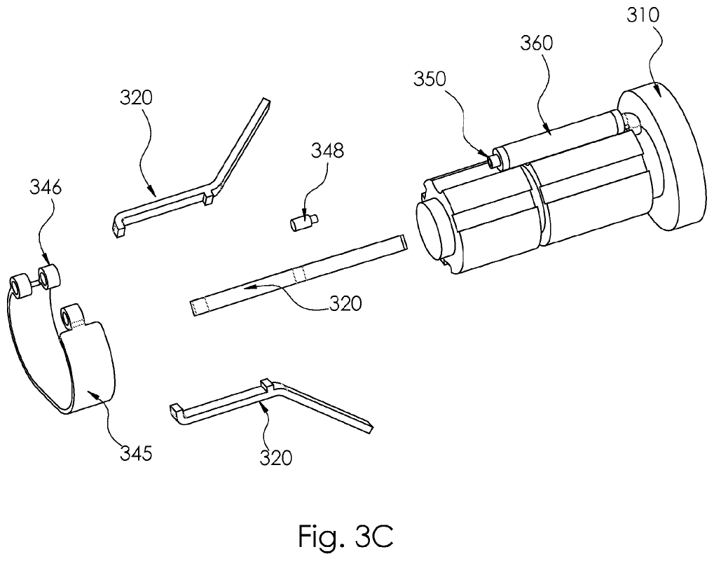

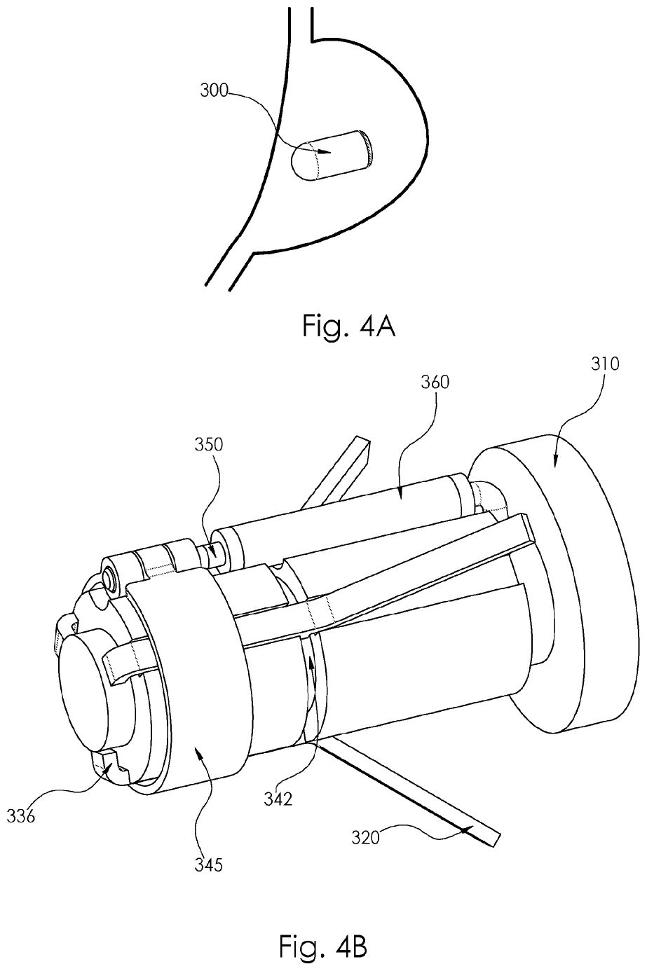

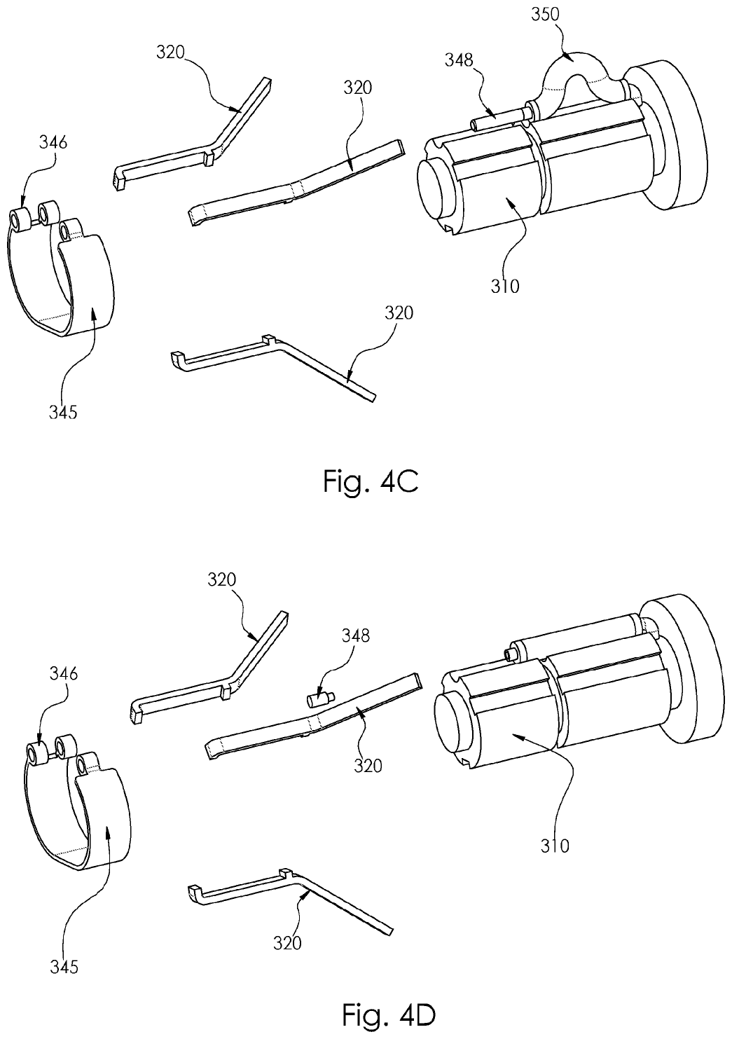

Reference is now made to FIGS. 3A, 3B and 3C, which are simplified respective assembled, sectional and exploded view illustrations of an in-vivo insertable device 300 constructed and operative in accordance with another preferred embodiment of the present invention. For clarity, reference is also made, in the course of the description which follows, to FIGS. 4A-4D.

It is to be appreciated that the in-vivo insertable device 300 may be any suitable in-vivo insertable device 300, preferably in the form of a capsule, which is suitable for insertion into an at least partially hollow organ of a human or animal and is intended for subsequent evacuation therefrom. Examples of suitable in-vivo insertable devices 300 include devices for drug perfusion, devices for in-vivo chemical analysis and devices for gastrointestinal tonometry. Reference is also made to U.S. Pat. Nos. 8,021,384 and 9,780,622 of the present applicant, the disclosures of which are hereby incorporated by reference.

The description which follows refers generally to an in-vivo insertable device 300 which is particularly suitable for insertion into a human stomach, it being understood that the present invention is not limited to this example.

Turning now to FIGS. 3A-3C, it is seen that in-vivo insertable device 300 preferably includes a main portion 310, which contains a payload 314, preferably comprising operative elements of the device 300. Examples of such operative elements include: electronics, batteries, motors, piezoelectric elements, controllers and wireless communication assemblies.

A plurality of device retaining elements 320 are preferably removably mounted onto main portion 310 and are retained in a retracted operative orientation by a capsule cover element 330, typically a gelatin capsule cover element which biodegrades upon being located within an organ, such as a stomach. FIG. 4A shows the device 300 just following location thereof within an organ and prior to biodegrading of capsule cover element 330.

Device retaining elements 320 preferably each include a main portion 332 and a wing portion 334 which is pre-stressed relative to the main portion 332, so as to assume the orientation shown in FIG. 4B, but is constrained by the capsule cover element 330, when intact, to assume the orientation shown in FIG. 3B. The wing portions 334 of the device retaining elements 320 are retained in a partially bent orientation by capsule cover member 330, as seen in FIG. 3B. Upon degradation of capsule cover member 330, the wing portions 334 of the device retaining elements 320 bend further outwardly and extend as seen in FIG. 4B.

Device retaining elements 320 also preferably include a forward inwardly directed retaining tab 336 and a rearward inwardly directed retaining tab 338, which are seated respectively at a location 340 forwardly of main portion 310 and in a circumferential slot 342 formed in main portion 310, in the operative orientations shown in FIGS. 4A and 4B. Reference to “forward” or “forwardly” in this description refers, in FIGS. 3A-4D to the left in these drawings.

Upon degradation of the capsule cover element 330, the device retaining elements 320 automatically bend further outwardly from the main portion 310, as seen in FIG. 4B. The automatic further bending preferably occurs due to pre-stressing of the device retaining elements 320. Device retaining elements 320 are attached to main portion 310 by a band 345 forming part of a fail-safe evacuation assembly, described hereinbelow. FIG. 4B shows the device in an operative orientation wherein the device 300 is retained within the organ by the spreading out of the device retaining elements 320.

It is a particular feature of a preferred embodiment of the present invention that the device 300 is provided with a fail-safe evacuation assembly, including mutually redundant mechanisms which enable evacuation of the device 300 from the organ by disassembly of the device retaining elements 320.

In accordance with a preferred embodiment of the present invention, the fail-safe evacuation assembly comprises split band 345, which is prestressed to an open operative orientation as seen in FIG. 3C. Split band 345 is formed with mutually axially arrangeable retaining tabs 346, which are retained in a mutually axial arrangement as seen in FIGS. 3B and 4B by a retaining rod assembly 347.

Preferably, retaining rod assembly 347 includes an engagement portion 348, which engages tabs 346 in the mutually axial arrangement shown in FIGS. 3B and 4B. Engagement portion 348 is preferably mounted onto a retractable retaining rod portion 350. In accordance with a preferred embodiment of the present invention, engagement portion 348 is biodegradable. This biodegradable engagement portion 348 provides one element of the fail-safe evacuation assembly.

Retractable retaining rod portion 350 is preferably formed to have a normally elongate rod portion 352 which terminates rearwardly in an inwardly-directed tab portion 354, which is retained in a recess 356 formed in main portion 310. Normally elongate rod portion 352 is preferably surrounded by a heating wire 358 which can alternatively be a heating sheet, and by a flexible insulation cover 360, made of one or more of plastic, rubber, shrink tubing and vacuum deposited polymer coating. Supply of electric current to heating wire 358, via a power source in the payload 314, typically in response to an actuation signal from a remote controller (not shown) outside of the body, causes heating of elongate rod portion 352 and resulting bending thereof due to a predetermined shape memory thereof as seen in FIG. 4C. This bending produces retraction of retaining rod portion 350.

It is appreciated that in the operative orientations shown in FIGS. 3B and 4B, split band 345 is retained by engagement portion 348 in a closed operative orientation and thus retains device retaining elements 320 in tight engagement against main portion 310 thus preventing disengagement of device retaining elements 320 from main portion 330 in the operative orientations shown in FIGS. 4A and 4B.

It is also appreciated that retractable retaining rod portion 350 provides another element of the fail-safe evacuation assembly 344, inasmuch as it can be retracted by heating wire 358, which can alternatively be a heating sheet, in response to actuation by a remote control via the payload 314.

Upon retraction of retractable retaining rod portion 350, engagement portion 348 is shifted rearwardly and out of engagement with band 345, allowing band to assume its open orientation as seen in FIG. 4C, and thus to release retaining tabs 336 and 338 of device retaining element 320 from engagement with main portion 310. The same result is achieved in a redundant manner by degradation of biodegradable engagement portion 348, which, when degraded, releases tabs 346 from the mutually axial arrangement shown in FIGS. 3B and 4B, and thus releases retaining tabs 336 and 338 of device retaining elements 320 from engagement with main portion 310, as seen in FIG. 4D.

As seen clearly in both of FIGS. 4C and 4D, this disengagement enables total disengagement of device retaining elements 320 from main portion 310 and enables the device retaining elements 320 and the main portion 310 and the various elements of the fail-safe evacuation assembly, to be readily evacuated from the organ, such as the stomach, by natural, biological evacuation processes. Each disengagement, by either of the two fail safe mechanisms, releases all of the retaining tabs 336 and 338 simultaneously, so all device retaining elements 320 will also be released simultaneously.

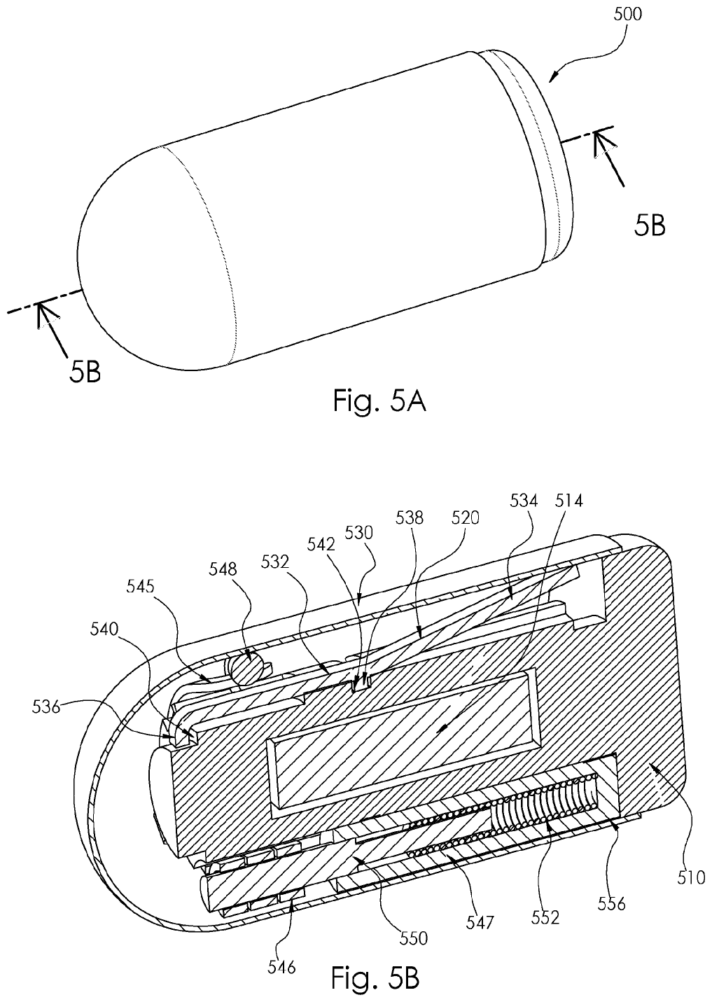

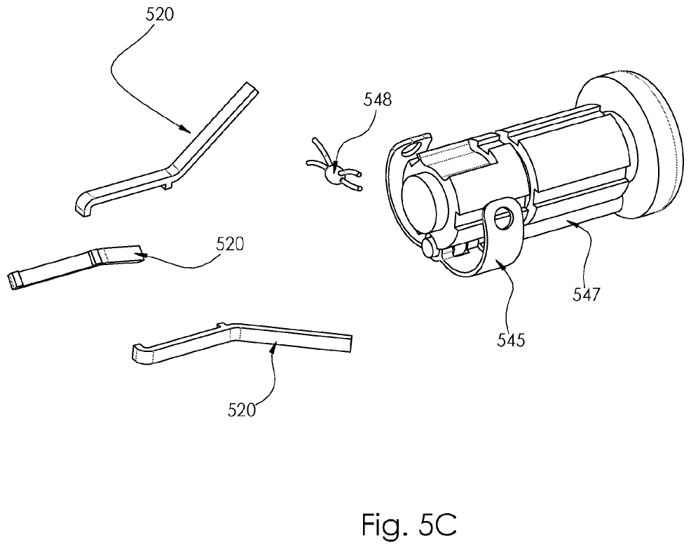

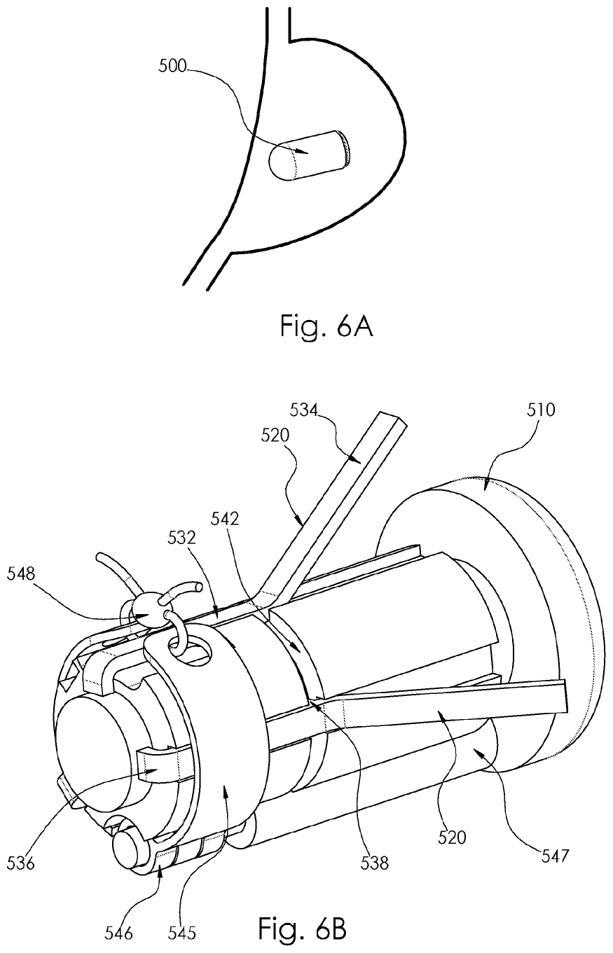

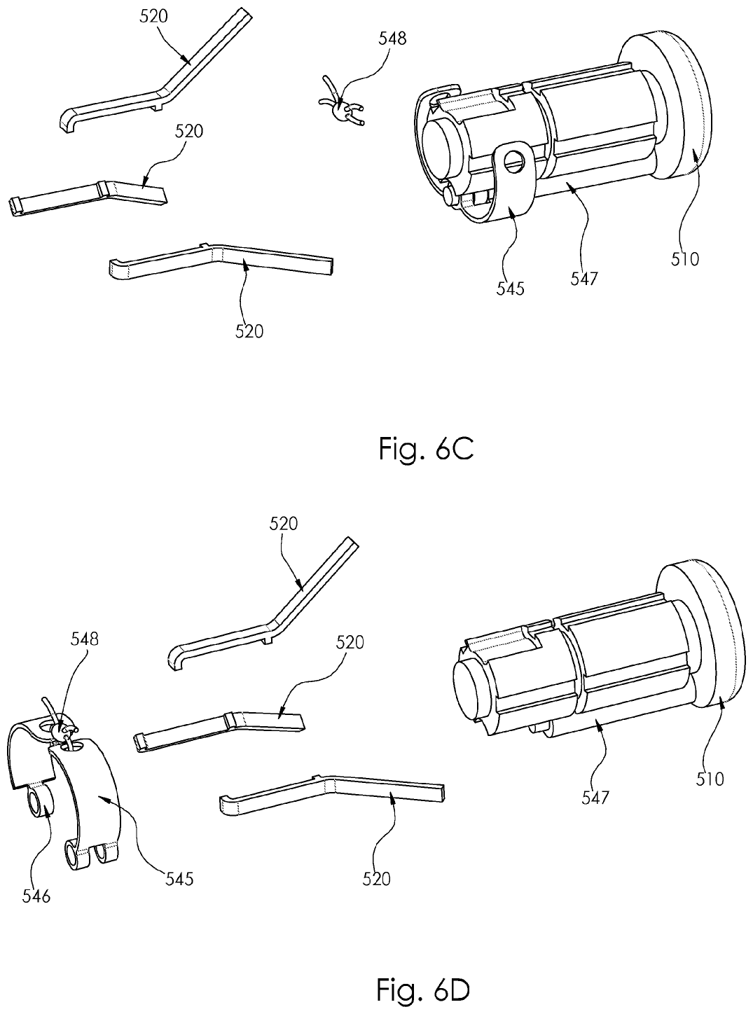

Reference is now made to FIGS. 5A, 5B and 5C, which are simplified respective assembled, sectional and exploded view illustrations of an in-vivo insertable device 500 constructed and operative in accordance with another preferred embodiment of the present invention. For clarity, reference is also made, in the course of the description which follows, to FIGS. 6A-6D.

It is to be appreciated that the in-vivo insertable device 500 may be any suitable in-vivo insertable device 500, preferably in the form of a capsule, which is suitable for insertion into an at least partially hollow organ of a human or animal and is intended for subsequent evacuation therefrom. Examples of suitable in-vivo insertable devices 500 include devices for drug perfusion, devices for in-vivo chemical analysis and devices for gastrointestinal tonometry. Reference is also made to U.S. Pat. Nos. 8,021,384 and 9,780,622 of the present applicant, the disclosures of which are hereby incorporated by reference.

The description which follows refers generally to an in-vivo insertable device 500 which is particularly suitable for insertion into a human stomach, it being understood that the present invention is not limited to this example.

Turning now to FIGS. 5A-5C, it is seen that in-vivo insertable device 500 preferably includes a main portion 510, which contains a payload 514, preferably comprising operative elements of the device 500. Examples of such operative elements include: electronics, batteries, motors, piezoelectric elements, controllers and wireless communication assemblies.

A plurality of device retaining elements 520 are preferably removably mounted onto main portion 510 and are retained in a retracted operative orientation by a capsule cover element 530, typically a gelatin capsule cover element which biodegrades upon being located within an organ, such as a stomach. FIG. 6A shows the device 500 just following location thereof within an organ and prior to biodegrading of capsule cover element 530.

Device retaining elements 520 preferably each include a main portion 532 and a wing portion 534 which is pre-stressed relative to the main portion 532, so as to assume the orientation shown in FIG. 6B, but is constrained by the capsule cover element 530, when intact, to assume the orientation shown in FIG. 5B.

Device retaining elements 520 also preferably include a forward inwardly directed retaining tab 536 and a rearward inwardly directed retaining tab 538, which are seated respectively at a location 540 forwardly of main portion 510 and in a circumferential slot 542 formed in main portion 510, in the operative orientations shown in FIGS. 5B and 6B. Reference to “forward” or “forwardly” in this description refers, in FIGS. 5A-6D, to the left in these drawings.

Upon degradation of the capsule cover element 530, the device retaining elements 520 automatically bend outwardly from the main portion 510. The automatic bending preferably occurs due to pre-stressing of the device retaining elements 520, which are held in their retracted operative orientation by a band forming part of a fail-safe evacuation assembly, described hereinbelow. FIG. 6B shows the device in an operative orientation wherein the device 500 is retained within the organ by the spreading out of the device retaining elements 520.

It is a particular feature of a preferred embodiment of the present invention that the device 500 is provided with a fail-safe evacuation assembly, including mutually redundant, mutually disparate, mutually independently operative mechanisms which enable evacuation of the device 500 from the organ by disassembly of the device retaining elements 520 from the main portion 510.

In accordance with a preferred embodiment of the present invention, the fail-safe evacuation assembly comprises a doubly split band 545, which is prestressed to an open operative orientation as seen in FIG. 5C. Doubly split band 545 is formed with mutually axially arrangeable retaining tabs 546, which are retained in a mutually axial arrangement as seen in FIGS. 5B and 6B by a retaining rod assembly 547. Split band 545 is also preferably retained in a closed operative orientation by a fastener 548.

Preferably, retaining rod assembly 547 engages tabs 546 in the mutually axial arrangement shown in FIG. 5B and includes a retractable retaining rod portion 550.

In accordance with a preferred embodiment of the present invention, fastener 548 is biodegradable. This biodegradable fastener provides one element of the fail-safe evacuation assembly.

Retractable retaining rod portion 550 is preferably surrounded by an electrically actuable coil 552, which is retained in a recess 556 formed in main portion 510. Coil 552 and rod portion 550 together define a solenoid. Supply of electric current to coil 552, via a power source in the payload 514, typically in response to an actuation signal from a remote controller (not shown) outside of the body, causes retraction of retaining rod portion 550 and disengagement thereof from split band 545.

It is appreciated that in the operative orientations shown in FIGS. 5B and 6B, split band 545 is retained by both retaining rod assembly 547 and by fastener 548 in a closed operative orientation and thus retains device retaining elements 520 in tight engagement against main portion 510, thus preventing disengagement of device retaining elements 520 from main portion 530 in the operative orientations shown in FIGS. 5B and 6B.

It is also appreciated that retaining rod assembly 547 provides another element of the fail-safe evacuation assembly, inasmuch as it can be retracted by a remote control via the payload 514.

Upon retraction of retractable retaining rod portion 550, retaining rod assembly 547 is displaced rearwardly and out of engagement with band 545, allowing band to assume its open orientation as seen in FIG. 6D, and thus to release retaining tabs 536 and 538 of device retaining element 520 from engagement with main portion 510. The same result is achieved in a redundant manner by degradation of biodegradable fastener 548, which, when degraded, releases band 545, which opens, as seen in FIG. 6C.

As seen clearly in both of FIGS. 6C and 6D, this disengagement enables total disengagement of device retaining elements 520 from main portion 510 and enables the device retaining elements 520 and the main portion 510 and the various elements of the fail-safe evacuation assembly, to be readily evacuated from the organ, such as the stomach, by natural, biological evacuation processes. Operation of either of the two fail safe mechanisms, releases all device retaining elements 520 simultaneously.

It will be appreciated by persons skilled in the art that the present invention is not limited to what has been particularly shown and described hereinabove. Rather the scope of the invention includes both combinations and subcombinations of features described hereinabove and modifications thereof which are not in the prior art.

1. An in-vivo insertable device, which is insertable into an at least partially hollow organ of a human or animal, comprising:
a housing formed of a material and being of a contour which enable it to be inserted into an at least partially hollow organ of a human or animal;
an operative portion disposed within said housing;
at least one device-retaining element having three operational states:
a first retracted state prior to and during insertion of said device;
a second expanded state operative to retain said operative portion within the at least partially hollow organ; and
a third disassembled state that enables said device to be naturally evacuated from the at least partially hollow organ,
said in-vivo insertable device being characterized in that it includes at least two mutually redundant, mutually diverse and mutually independently operative disassembly mechanisms which cause said at least one device-retaining element to shift to said third disassembled state.

2. An in-vivo insertable device according to claim 1 and wherein said at least one device-retaining element comprises at least two device-retaining elements and wherein said at least two mutually redundant, mutually diverse and mutually independently operative disassembly mechanisms cause said at least two device-retaining elements to shift from said second expanded state to said third disassembled state simultaneously.

3. An in-vivo insertable device according to claim 1 and wherein said at least one device-retaining element comprises a first remotely actuable mechanism and a second automatically operable mechanism.

4. An in-vivo insertable device according to claim 3 and wherein said second automatically operable mechanism is one of a biodegradable, bioabsorbable and bioresorbable element which biodegrades within said organ and thereby causes said shift to said third disassembled state.

5. An in-vivo insertable device according to claim 3 and wherein said first remotely actuable mechanism includes an electromagnetic actuator.

6. An in-vivo insertable device according to claim 3 and wherein said first remotely actuable device includes a heat responsive shape changing element.

7. An in-vivo insertable device according to claim 3 and wherein said first remotely operable mechanism is actuatable by a remote controller outside of said human or animal.

8. An in-vivo insertable device according to claim 3 and wherein said second automatically operable mechanism is actuable by a controller included within said operative portion in accordance with a preplanned program.

9. An in-vivo insertable device according to claim 3 and wherein said first remotely actuable device includes a heat responsive displaceable element having a shape memory, which undergoes heating in response to remote actuation.

10. An in-vivo insertable device according to claim 9 and wherein in said first remotely actuable mechanism said heating is achieved by connecting electric contacts directly to said heat responsive displaceable element having a shape memory, which serves also as a heating element.

11. An in-vivo insertable device according to claim 1 and wherein at least one of said at least two mutually redundant, mutually diverse and mutually independently operative disassembly mechanisms employ at least one of magnetic forces, electric current, electrostatic forces, external pressure and a heating element.

12. An in-vivo insertable device according to claim 9 and wherein said heating is triggered by a controller and achieved by connecting batteries, located in said payload, to said heat responsive displaceable element.

13. An in-vivo insertable device according to claim 1 and wherein all dissembled elements of said device are formed with rounded and smooth edges so that they will not harm any parts of a hollow organ during evacuation.

14. An in-vivo insertable device according to claim 3 and wherein at least one of said at least two mutually redundant, mutually diverse and mutually independently operative disassembly mechanisms can be actuated via endoscopy.

15. An in-vivo insertable device according to claim 3 and wherein at least one of said at least two mutually redundant, mutually diverse and mutually independently operative disassembly mechanisms comprises at least one of a split band and a connection part which holds said device retaining elements onto the main element, and which is operative when said at least one mechanism is actuated to release said device retaining elements, so that the device and all its parts are free to evacuate the hollow organ.

16. An in-vivo insertable device according to claim 9 and wherein the heat responsive displaceable element is heated by at least one of a heating micro-wire and a heating sheet.

17. An in-vivo insertable device according to claim 16 and wherein the at least one of a heating micro-wire and a heating sheet is covered by flexible insulation cover made of one or more of plastic, rubber, shrink tubing and vacuum deposited polymer coating.

18. An in-vivo insertable device, which is insertable into an at least partially hollow organ of a human or animal, comprising:
a housing formed of a material and being of a contour which enable it to be inserted into an at least partially hollow organ of a human or animal;
an operative portion disposed within said housing;
at least one device-retaining element having three operational states:
a first retracted state prior to and during insertion of said device;
a second expanded state operative to retain said operative portion within the at least partially hollow organ; and
a third disassembled state that enables said device to be naturally evacuated from the at least partially hollow organ,
said in-vivo insertable device being characterized in that it includes at least two mutually redundant and mutually independently operative disassembly mechanisms which cause said at least one device-retaining element to shift to said third disassembled state.
